(19) United States
(12) Patent Application Publication
(10) Pub. No.: US 2024/0180443 A1
(43) Pub. Date: Jun. 6, 2024

(54) Sample Collection Device and Method

(71) Applicant: 3M INNOVATIVE PROPERTIES COMPANY, St. Paul, MN (US)

(72) Inventors: Audrey A. Sherman, Woodbury, MN (US); Brett J. Sitter, Marine On St. Croix, MN (US); Alan R. Dombrowski, Woodbury, MN (US); Laura R. Nereng, Woodbury, MN (US); Michael R. Berrigan, Oakdale, MN (US); Narina Y. Stepanova, Woodbury, MN (US)

(21) Appl. No.: 18/553,589

(22) PCT Filed: Mar. 29, 2022
(86) PCT No.: PCT/IB2022/052903

§ 371 Date: Oct. 2, 2023

Related U.S. Application Data
(60) Provisional application No. 63/200,901, filed on Apr. 2, 2021.
(60) Provisional application No. 63/227,519, filed on Jul. 30, 2021.

(51) Int. Cl. A61B 5/08; A61B 5/097; A61B 10/00; G01N 33/543; G01N 33/569

(52) U.S. Cl. CPC A61B 5/082; A61B 5/097; A61B 10/00; G01N 33/54388; G01N 33/54391; G01N 33/569; A61B 2010/0087; G01N 2800/12

(57) ABSTRACT

The present disclosure provides a sample collection device. The sample collection device includes a housing defining a fluid channel from a first portion to a second portion. The sample collection device further includes a porous sample collection media disposed within the housing and in fluid communication with the fluid channel. The sample collection device further includes a fluid inlet port disposed in fluid communication with the porous sample collection media. The fluid inlet port is configured to direct a test fluid onto the porous sample collection media. The sample collection device further includes an assay configured to receive a fluid that was incident on the porous sample collection media.

### TECHNICAL FIELD

The present disclosure relates generally to sample collection devices and systems as well as methods for using the sample collection devices and systems. The present disclosure also relates to aerosol sample collection devices and systems as well as methods for using the aerosol sample collection devices and systems.

### BACKGROUND

As Covid-19 reached pandemic status, increased availability of diagnostic testing was important to help identify and control the serious illness. This illness has highlighted the need for widespread availability of such diagnostic tests even after the pandemic ends. Diagnostic tests typically require a nasopharyngeal swab involving insertion of a 6-inch-long swab into the back of the nasal passage through one nostril and rotation of the swab for approximately 15 seconds. This process is then repeated with the other nostril. The swab is then inserted into a clean container and sent to a lab for processing. Other nasal swab tests require sampling from the mid-turbinate area of the nasal passage—again sampled from both nostrils. Still others require sampling from the anterior nares in both nostrils. Other diagnostic tests involve collecting a saliva sample and then placing it in a clean container and sending it to a lab for processing. Currently available at-home viral (for example, Covid-19) tests involve a nasal swab as a described above but do not require sending to a lab for processing (for example, the Ellume™ test, the Abbot™ BinaxNOW™ test, and the Lucira™ All-in-One test kit). Tests that utilize nasal swab samples or saliva contend with contaminants that can interfere with the various diagnostic tests. As a result, these sample types require a purification step when using RT-PCR molecular testing.

### SUMMARY

The inventors of the present disclosure recognized that the sample collection devices and test processes described above have various challenges. For example, most of the available tests require that the collection device be processed at a laboratory, increasing cost and delaying delivery of results. Further, many of the test methods require that the sample collection mechanism be a nasopharyngeal or other type of nasal or oral swab, which is uncomfortable for the user. This discomfort can cause users to opt out of testing. Further, there may be possibility of contamination of the sample during transfer to the clean container, removal from the container, etc. Due to the multiple steps and devices involved and the possibility of contamination of the sample, such conventional methods and devices for sample collection and eluent testing may be used by only trained professionals (e.g., medical personnel), and may be complicated for use by a user with little or no training.

As such, the inventors of the present disclosure sought to create easy-to-use, inexpensive integrated sample collection and testing devices in which sample collection and sample testing happen in a single device that can be used in any location by a layperson. Additionally, the inventors sought to create an integrated sample collection and testing device that did not require the user to undergo a nasopharyngeal swab.

Thus, the inventors of the present disclosure invented the sample collection and testing devices and methods described herein. In these devices, a single unit collects a sample airflow and tests a corresponding eluent to detect the presence of pathogens in the sample airflow. Moreover, the assay is in contact with the porous sample collection media to receive an eluent from the porous sample collection media. The sample collection and testing devices described herein enable rapid testing of a sample airflow and provide increased efficiency and decreased a cost and complexity. Moreover, the disclosed sample collection devices may minimize a possibility of contamination of the sample since both sample collection and testing are performed in a single unit. Further, the disclosed sample collection device may be easily used by a user (e.g., a potential patient) without any prior training or professional help.

In some embodiments, the sample collection device is a two-part assembly, where the two parts are detachably coupled to each other. Specifically, one of the parts includes (1) an air or bioaersol exhalation portion or mouthpiece portion and (2) an air outlet portion, and the other one of the parts includes a porous sample collection media and an assay. The assay is configured to receive an eluent from the porous sample collection media. The two-part design of the sample collection device may facilitate a replacement of one of the parts including the porous sample collection media and the assay, whenever the porous sample collection media and/or assay may need to be replaced. Therefore, whenever there is a need for replacement, a user can easily replace a used porous sample collection media and/or a used assay from the sample collection device with a new porous sample collection media and/or a new assay.

Some embodiments of the present disclosure relate to a sample collection device including: a housing extending from a first portion to a second portion, the housing defining a fluid channel from the first portion to the second portion, wherein the first portion is configured to receive an exhalation airflow; a porous sample collection media disposed within the housing and in fluid communication with the fluid channel; a fluid inlet port defining a hole through the housing and disposed in fluid communication with the porous sample collection media, wherein the fluid inlet port is configured to direct a test fluid onto the porous sample collection media; and an assay disposed within the housing and contacting the porous sample collection media, wherein the assay is configured to receive a fluid from the porous sample collection media.

In some embodiments, the housing further includes a barrier disposed between the fluid channel and the assay, and wherein the barrier is configured to prevent direct fluid communication between the exhalation airflow and the assay.

In some embodiments, the barrier further includes an opening configured to receive the porous sample collection media therethrough. In some embodiments, the housing further includes a receptacle disposed in fluid communication with the opening and configured to receive the assay therein.

In some embodiments, the housing includes: a first housing part including the first portion and the second portion, the first housing part further defining the fluid channel from the first portion to the second portion; and a second housing part engaged with the first housing part and including the fluid inlet port, wherein the porous sample collection media and the assay are disposed within the second housing part, the second housing part further defining one or more apertures disposed in fluid communication with the porous sample collection media and configured to allow the exhalation airflow to pass through the porous sample collection media.

In some embodiments, the first housing part further includes a first slot therethrough and a second slot therethrough aligned with the first slot, wherein each of the first slot and the second slot is configured to slidably receive the second housing part therethrough. In some embodiments, the second housing part is slidably and removably received within the first housing part along an insertion direction, wherein the second housing part further includes a stop element configured to abut the first housing part and prevent further movement of the second housing part relative to the first housing part along the insertion direction.

In some embodiments, the second housing part has a first end and a second end opposite to the first end, wherein: the fluid inlet port is disposed at the first end; the assay is disposed proximal to the second end; and the porous sample collection media is at least partly disposed between the fluid inlet port and the assay.

In some embodiments, the one or more apertures include one or more first apertures and one or more second apertures, wherein the second housing part further includes a first wall facing the first portion and defining the one or more first apertures therethrough, and a second wall opposite to the first wall and defining the one or more second apertures therethrough, and wherein each of the porous sample collection media and the assay is disposed between the first wall and the second wall.

In some embodiments, the porous sample collection media is at least partly disposed between the one or more first apertures and the assay to prevent direct fluid communication between the exhalation airflow and the assay.

In some embodiments, the second housing part further includes a coupling portion including the fluid inlet port, wherein the coupling portion is configured to be interchangeably and removably coupled with the first housing part and the metered fluid dose element, wherein the coupling portion further includes one or more apertures of the second housing part disposed in fluid communication and aligned with the fluid inlet port, and wherein the one or more apertures are configured to direct the test fluid from the fluid inlet port to the porous sample collection media when the metered fluid dose element is coupled with the coupling portion.

In some embodiments, the coupling portion further includes a shoulder configured to be detachably coupled with the first housing part, wherein the fluid channel of the first housing part is disposed in fluid communication with the one or more apertures upon coupling of the first housing part with the coupling portion. In some embodiments, the second housing part includes a longitudinal axis along its length, wherein the assay is spaced apart from the one or more apertures along the longitudinal axis to prevent direct fluid communication between the exhalation airflow and the assay. In some embodiments, the coupling portion further includes an internal surface at least partially defining the one or more apertures and one or more support members connecting the fluid inlet port with the internal surface.

In some embodiments, the housing further includes a display window configured to allow visual inspection of at least a portion of the assay.

In some embodiments, the sample collection device further includes a screen disposed in the housing and upstream of the porous sample collection media, wherein the screen includes one or more flow apertures therethrough.

In some embodiments, the housing further includes one or more vents disposed in fluid communication with the fluid channel and downstream of the porous sample collection media, wherein the one or more vents are configured to allow egress of fluid from the fluid channel.

In some embodiments, the fluid inlet port includes a protrusion extending away from an outer surface of the housing.

In some embodiments, the metered fluid dose element is movably attached to the fluid inlet port via a threaded connection.

In some embodiments, the metered fluid dose element contains a fluid reservoir.

In some embodiments, the porous sample collection media includes a surface area and the fluid reservoir includes a volume, wherein the volume divided by the surface area is in a range from 10 microliters/cm2 to 400 microliters/cm2, or from 10 microliters/cm2 to 250 microliters/cm2. In some embodiments, the volume of the fluid reservoir is in a range from 50 microliters to 500 microliters.

In some embodiments, the porous sample collection media includes a nonwoven filtration layer having an electrostatic charge. In some embodiments, the nonwoven filtration layer is hydrophobic.

In some embodiments, the test fluid is an aqueous solution including a surfactant.

In some embodiments, the assay includes an L-shaped recess configured to at least partially receive the porous sample collection media therein.

In some embodiments, the assay is fixedly attached to the porous sample collection media. In some embodiments, the assay detects virus or pathogen presence in the exhalation airflow and/or the test fluid. In some embodiments, the assay is a lateral flow assay. In some embodiments, the assay is a vertical flow assay.

Some embodiments of the present disclosure relate to a method for testing an exhalation airflow, the method including: coupling a porous sample collection media with an assay, such that the assay is configured to receive a fluid from the porous sample collection media; receiving the porous sample collection media and the assay within a housing, the housing defining a fluid channel configured to receive the exhalation airflow; flowing the exhalation airflow through the porous sample collection media, wherein the porous sample collection media is disposed in fluid communication with the fluid channel and forms a loaded porous sample collection media; flowing, a test fluid through the loaded porous sample collection media disposed in the fluid channel forming an eluent; and collecting and testing, by the assay, the eluent.

In some embodiments, the test fluid is a metered dose of test fluid. In some embodiments, the test fluid is delivered by a metered fluid dose element. In some embodiments, flowing the metered dose of the test fluid includes flowing a metered dose in a range from 50 microliters to 400 microliters of the test fluid through the loaded porous sample collection media disposed in the fluid channel.

In some embodiments, testing the eluent includes detecting a presence of virus or pathogen in the eluent.

In some embodiments, the method further includes detachably connecting the metered fluid dose element to a fluid inlet port of the housing.

In some embodiments, the housing includes a first housing part defining the fluid channel and a second housing part separate from the first housing part and configured to receive the porous sample collection media and the assay therein. In some embodiments, the method further includes detachably coupling the first housing part with the second housing part.

In some embodiments, the method further includes removing the first housing part from the second housing part; and detachably coupling the metered fluid dose element to the second housing part.

Some embodiments of the present disclosure provide a sample collection device that does not require the user to undergo a nasopharyngeal or other nasal or oral swab.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a schematic front perspective view of a sample collection device, according to an embodiment of the present disclosure.

FIG. 2 is a schematic sectional side view of the sample collection device of FIG. 1, according to an embodiment of the present disclosure.

FIG. 3A is a schematic side view of an assay of the sample collection device of FIG. 1, according to an embodiment of the present disclosure.

FIG. 3B is a schematic front view of an assay of the sample collection device of FIG. 1, according to another embodiment of the present disclosure.

FIG. 4 is a schematic rear perspective view of a housing of the sample collection device of FIG. 1, according to an embodiment of the present disclosure.

FIG. 5 is a schematic front perspective view of another sample collection device, according to an embodiment of the present disclosure.

FIG. 6 is a schematic perspective view of a first housing part of a housing of the sample collection device of FIG. 5, according to an embodiment of the present disclosure.

FIG. 7A is a schematic sectional side view of a second housing part of the housing of the sample collection device of FIG. 5, according to an embodiment of the present disclosure.

FIG. 7B is a schematic perspective view of the second housing part of FIG. 7A showing internal components, according to an embodiment of the present disclosure.

FIG. 7C is a schematic front perspective view of the second housing part of FIG. 7A, according to an embodiment of the present disclosure.

FIG. 7D is a schematic rear perspective view of the second housing part of FIG. 7A, according to an embodiment of the present disclosure.

FIG. 8 illustrates a schematic perspective view of a porous sample collection media and an assay of the sample collection device of FIG. 5, according to an embodiment of the present disclosure.

FIG. 9 is a schematic front perspective view of another sample collection device, according to an embodiment of the present disclosure.

FIG. 10 is a schematic side perspective view of the sample collection device of FIG. 9, according to an embodiment of the present disclosure; and

FIG. 11 is a flowchart for a method for testing an exhalation airflow, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

The term “substantially” as used here has the same meaning as “significantly,” and can be understood to modify the term that follows by at least about 90%, at least about 95%, or at least about 98%.

The term “about” is used here in conjunction with numeric values to include normal variations in measurements as expected by persons skilled in the art and is understood have the same meaning as “approximately” and to cover a typical margin of error, such as +5% of the stated value.

Terms such as “a,” “an,” and “the” are not intended to refer to only a singular entity but include the general class of which a specific example may be used for illustration.

The terms “a,” “an,” and “the” are used interchangeably with the term “at least one.” The phrases “at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have”, “having”, “include”, “including”, “comprise”, “comprising” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method or the like, means that the components of the composition, product, method or the like are limited to the enumerated components and any other components that do not materially affect the basic and novel characteristic(s) of the composition, product, method or the like.

Any direction referred to here, such as “front,” “back,” “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

The terms “downstream” and “upstream” refer to a relative position based on a direction of exhalation airflow through the device. Such exhalation can come from the user's mouth or nose. In other words, the bioaerosol exhalation can be oral or nasal.

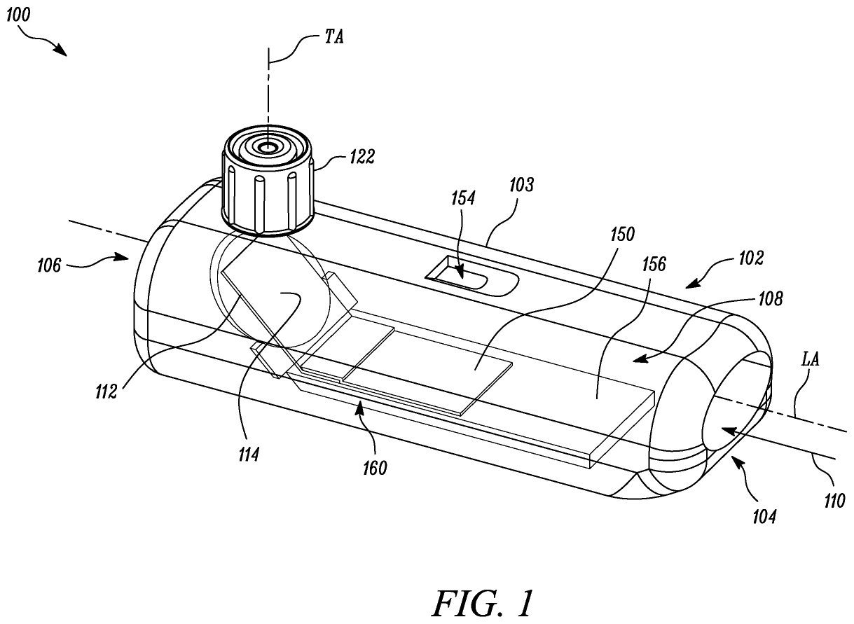

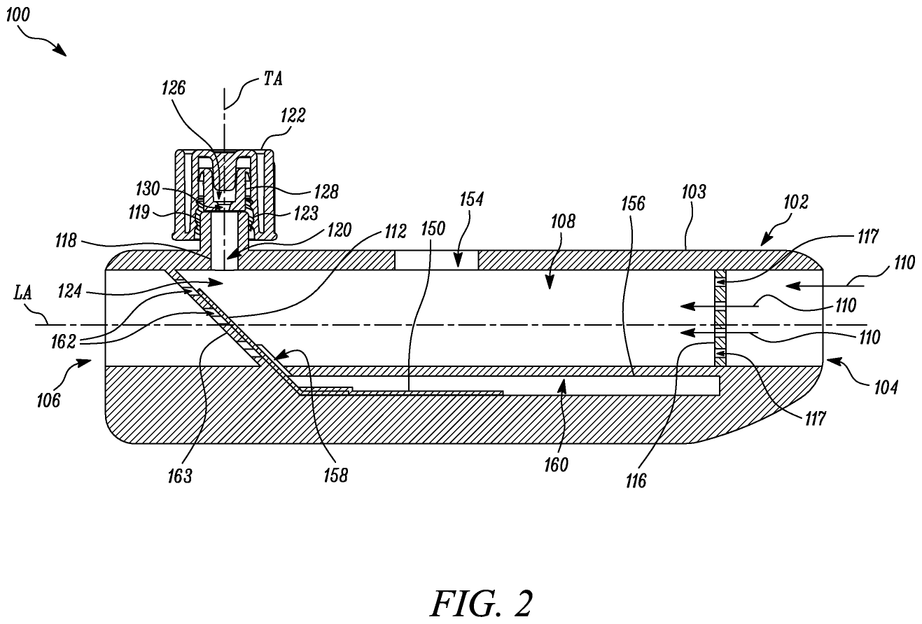

Referring now to Figures, FIGS. 1 and 2 illustrate different views of a sample collection device 100, in accordance with an embodiment of the present disclosure. The sample collection device 100 includes a housing 102 extending from a first portion 104 to a second portion 106. The housing 102 is shown transparent in FIG. 1 for illustrative proposes only. The housing 102 includes a fluid channel 108 from the first portion 104 to the second portion 106. In some embodiments, one of the first portion 104 and the second portion 106 is a mouthpiece portion and the other of the first portion 104 and the second portion 106 is an air outlet portion. As used herein, the mouthpiece portions relates to a device into which the user may breathe (exhalation or inhalation) through the user's nose or mouth. The term “mouthpiece” is not meant to be limited to the user's oral breath or breath through the user's mouth.

In the illustrated embodiment of FIG. 1, the first portion 104 is a mouthpiece portion and the second portion 106 is an air outlet portion. The first portion 104 is configured to receive an exhalation airflow 110. In some other embodiments, the second portion 106 may receive the exhalation airflow 110. In that case, the first portion 104 is an air outlet portion and the second portion 106 is a mouthpiece portion. The first portion 104 and the second portion 106 may be interchangeably used as the mouthpiece portion and the air outlet portion based on application requirements. The housing 102 may be formed of a rigid material, such as plastic. As used herein, the term “fluid” may refer to a gas (i.e., air).

In some embodiments, the first portion 104 and the second portion 106 may be integral parts of the housing 102. In some embodiments, one or both of the first portion 104 and the second portion 106 may be separate parts that can be detached from the housing 102.

A user exhales into the first portion 104 to introduce the exhalation airflow 110. Thus, in the illustrated embodiment of FIGS. 1 and 2, the fluid channel 108 is an air flow channel extending from the first portion 104 to the second portion 106. The fluid channel 108 extends longitudinally along a longitudinal axis “LA” of the housing 102, as shown in FIGS. 1 and 2.

With reference to FIGS. 1 and 2, the sample collection device 100 further includes a porous sample collection media 112 disposed within the housing 102 and in fluid communication with the fluid channel 108. The porous sample collection media 112 may be disposed or fixed along the fluid channel 108. The porous sample collection media 112 may be replaceable and changed out by a user, as desired. For example, a user may exhale, via the first portion 104, into the sample collection device 100 and load the porous sample collection media 112 with a sample of the exhalation airflow 110 to form a loaded porous sample collection media 112. The user may then test the loaded porous sample collection media 112. The testing may take place with the loaded porous sample collection media 112 located in place (i.e., in situ) within the sample collection device 100. After conducting the test, the user may replace the loaded porous sample collection media 112 with an unloaded porous sample collection media 112. While the porous sample collection media 112 is illustrated herein as defining a planar element, it is understood that the porous sample collection media 112 may define any shape when disposed within the housing 102 and along the fluid channel 108. In some embodiments, the porous sample collection media 112 is three dimensional.

The porous sample collection media 112 at least partially occludes the fluid channel 108. In the illustrated embodiment of FIGS. 1 and 2, the porous sample collection media 112 has a major plane 114 forming an angle with a direction of the exhalation airflow 110 passing through a thickness of the porous sample collection media 112. In some embodiments, this angle may be in a range from about 91 degrees to about 179 degrees, or from about 100 degrees to about 160 degrees, or about 115 degrees to about 150 degrees, or about 125 to about 145 degrees. In some other embodiments, the porous sample collection media 112 may have the major plane 114 that is substantially orthogonal to the direction of the exhalation airflow 110 passing through the thickness of the porous sample collection media 112.

In some cases, the porous sample collection media 112 may have a thickness (orthogonal to the major plane 114) in a range from 200 micrometers (mm) to 1000 mm, or from 250 mm to 750 mm. In some embodiments, the porous sample collection media 112 includes a surface area (not shown), which can be interchangeably referred to as “major plane surface area”. In some cases, the porous sample collection media 112 may have a major plane surface area in a range from about 1 cm² to about 4 cm², or about 2 cm² to about 3 cm².

The porous sample collection media 112 may be a nonwoven material configured to filter or capture pathogens or virus from an exhalation airflow. In some embodiments, the porous sample collection media 112 includes a nonwoven filtration layer having an electrostatic charge. Specifically, the porous sample collection media 112 includes a nonwoven filtration layer having an electrostatic charge configured to filter pathogens from the exhalation airflow 110. In some embodiments, the nonwoven filtration layer is hydrophobic. Thus, the hydrophobic nonwoven filtration layer is configured to filter pathogens from the exhalation airflow 110.

The sample collection media may be pleated. In some embodiments, the pleat frequency is between about 1 pleat per 0.6 cm of media and about 1 pleat per 2 mm of media. In some embodiments, the pleat height is between about 2 mm and about 4 mm.

The term “hydrophobic” refers to a material having a water contact angle of 90 degrees or greater, or from about 90 degrees to about 170 degrees, or from about 100 degrees to about 150 degrees. Water contact angle is measured using ASTM D5727-1997 Standard test method for surface wettability and absorbency of sheeted material using an automated contact angle tester.

In some cases, the porous sample collection media 112 may be formed of a polymeric material. In some cases, the porous sample collection media 112 may be formed of a polyolefin. For example, the porous sample collection media 112 may be formed of polypropylene. Exemplary nonwovens for use in or as the porous sample collection media 112 include, for example, those described in U.S. Pat. Nos. 7,947,142; 8,162,153; 9,139,940; and 10,273,612, all of which are incorporated herein in their entirety.

The porous sample collection media may be formed of a of a polylactide (PLA) such as, for example, 6100D from NatureWorks LLC15305 Minnetonka Blvd Minnetonka, MN 55345. Exemplary nonwoven filtration layer materials for use in or as the porous sample collection media include, for example, those described in U.S. Pat. Nos. 7,947,142; 8,162,153; 9,139,940; and 10,273,612, all of which are incorporated herein in their entirety.

Referring to FIG. 2, in some embodiments, the sample collection device 100 further includes a pre-filter or screen 116 disposed in the housing 102 and upstream of the porous sample collection media 112. Screen 116 catches solid material or debris (such as, for example, food particles) and catches them so that they are not incident on the porous sample collection media 112. The screen 116 is not shown in FIG. 1 for illustrative purposes. In some cases, the screen 116 may be a pre-filter. The screen 116 may be fixed within the housing 102 and disposed between the first portion 104 (i.e., mouthpiece portion) and the porous sample collection media 112. In some embodiments, the screen 116 includes one or more flow apertures 117 therethrough. The exhalation airflow 110 passes through a thickness of the screen 116. The screen 116 at least partially occludes the fluid channel 108. In some cases, the screen 116 may have a major plane (not shown) that is orthogonal to the direction of the exhalation airflow 110 passing through the thickness of the screen 116. The screen 116 may be a non-woven layer configured to filter out larger particles from the exhalation airflow 110 passing through the screen 116. In some cases, the screen 116 may be a non-woven layer that does not have an electrostatic charge. The screen 116 may not capture pathogens and may allow them to transmit through the screen 116. In some embodiments, the screen is made of or includes at least one of a plastic mesh, a woven net, a needle tacked fibrous web, a knitted mesh, an extruded net, and/or a carded or spunbond coverstock. In some embodiments, the screen does not catch or remove from the airflow particles having a size of less than 100 micrometers, or 75 micrometers, or 50 micrometers, or 25 micrometers, or 10 micrometers, or 5 micrometers.

With reference to FIG. 2, the sample collection device 100 further includes a fluid inlet port 118. The fluid inlet port 118 includes a hole 120 through the housing 102 and disposed in fluid communication with the porous sample collection media 112. Specifically, the fluid inlet port 118 is adjacent to the porous sample collection media 112. In some embodiments, the fluid inlet port 118 includes a protrusion 119 extending away from an outer surface 103 of the housing 102.

The fluid inlet port 118 is configured to direct a test fluid onto the porous sample collection media 112. Specifically, as shown in FIG. 2, the fluid inlet port 118 is configured to direct the test fluid onto the porous sample collection media 112 via a flow passage 124 defined by the housing 102. The flow passage 124 may deliver the test fluid, via capillary action, to the porous sample collection media 112.

In some embodiments, the test fluid is an aqueous solution comprising a surfactant. In some embodiments, the test fluid may be an aqueous buffer solution. In some embodiments, the test fluid may be a saline solution. In some embodiments, the test fluid may be a saline solution including a surfactant. In some embodiments, the test fluid may be a saline solution including from about 0.5% to about 2% surfactant by weight.

In some embodiments, the sample collection device 100 further includes a metered fluid dose element 122 attached to the fluid inlet port 118. The metered fluid dose element 122 is configured to dispense a metered dose of the test fluid into the fluid inlet port 118. In some embodiments, the metered fluid dose element 122 is movably attached to the fluid inlet port 118 via a threaded connection 123. In other words, the metered fluid dose element 122 is detachably connected to the fluid inlet port 118 of the housing 102. Specifically, the metered fluid dose element 122 is movably attached to the protrusion 119 defined by the fluid inlet port 118. Thus, the metered fluid dose element 122 may be moved between a fluid loaded position and a fluid depleted position by rotating the metered fluid dose element 122 about a threaded axis “TA”. In some embodiments, the metered fluid dose element 122 is removably attached to the fluid inlet port 118 by other mechanical arrangements. In some other embodiments, the metered fluid dose element 122 may be an integral part of the fluid inlet port 118. In some embodiments, the metered fluid dose element 122 may be a replaceable element connected to the fluid inlet port 118.

In other embodiments, the sample collection device 100 does not include a metered fluid dose element. Instead, fluid is dispensed by the user into/onto the sample collection device in the area shown as including the metered dose element 122. Fluid can be added or dispensed as is commonly known including by use of a dropper or bottle.

As shown in FIGS. 1 and 2, in some cases, the metered fluid dose element 122 may extend orthogonally from the housing 102. In some other embodiments, the metered fluid dose element 122 may extend from the housing 102 at any oblique angle. The metered fluid dose element 122 may define a shape of an end cap that dispenses a metered dose of test fluid upon movement of the metered fluid dose element 122 towards the housing 102. In some cases, the metered fluid dose element 122 may define any other shape. In some cases, the metered fluid dose element 122 may be deformable and configured to discharge the test fluid upon squeezing of the deformable surface (not shown) of the metered fluid dose element 122 by a user.

Referring to FIG. 2, in some embodiments, the metered fluid dose element 122 contains a fluid reservoir 126 defining a volume to store a test fluid. The metered fluid dose element 122 is configured to release the test fluid from the fluid reservoir 126 as the metered fluid dose element 122 is moved from the fluid loaded position to the fluid depleted position. The metered fluid dose element 122 further includes a plunger element 128 that moves into the volume of the fluid reservoir 126 to release the test fluid as the metered fluid dose element 122 is moved from the fluid loaded position to the fluid depleted position. The plunger element 128 includes an aperture 130 to release the test fluid from the fluid reservoir 126 as the metered fluid dose element 122 is moved from the fluid loaded position to the fluid depleted position. One illustrative example of the metered fluid dose element 122 is commercially available from 3M Company (St. Paul MN, U.S.A.) under the trade designation CUROS™. Exemplary metered fluid dose elements include, for example, those described in U.S. Pat. Nos. 10,617,780 and 9,907,617 and in PCT Patent Application Publication No. WO2020/170169, all of which are incorporated herein in their entirety.

In some embodiments, the volume of the fluid reservoir 126 is in a range from 50 microliters to 500 microliters. In some embodiments, the volume divided by the surface area (i.e., a surface area of the porous sample collection media 112) is in a range from 10 microliters/cm2 to 400 microliters/cm2, or from 10 microliters/cm2 to 250 microliters/cm2. The surface area of the porous sample collection media 112 may correspond to an area of a major surface of the porous sample collection media 112. Specifically, the surface area of the porous sample collection media 112 may correspond to an area defined by the porous sample collection media 112 in the major plane 114.

Where present, the metered fluid dose element 122 dispenses a metered dose of the test fluid through the loaded porous sample collection media 112 disposed in the fluid channel 108 to form an eluent. In other words, the test fluid may be delivered through the fluid inlet port 118 and applied onto the loaded porous sample collection media 112 to form the eluent. In some embodiments, a metered dose of the test fluid is in a range from 50 microliters to 400 microliters of the test fluid flowing through the loaded porous sample collection media 112 disposed in the fluid channel 108. Further, the test fluid may travel through a surface and a thickness of the loaded porous sample collection media 112 carrying any pathogen that is present on the loaded porous sample collection media 112. The eluent is then collected and tested, as described later in the present disclosure.

In some cases, the sample collection device 100 may include a pressing element (not shown) that is configured to apply pressure onto the loaded porous sample collection media 112. The pressing element may force a remaining test fluid out of the loaded porous sample collection media 112 for collection and testing. The pressing element may be attached to the metered fluid dose element 122 where movement of the metered fluid dose element 122 actuates the pressing element onto the loaded porous sample collection media 112.

In some embodiments, the sample collection device 100 may include two or more fluid inlet ports 118. Each fluid inlet port 118 may independently include a metered fluid dose element 122 movably attached to the corresponding fluid inlet port 118. Each of the two or more metered fluid dose elements 122 may include the same test fluid. Alternatively, at least one of the two or more metered fluid dose elements 122 may include a test fluid that is different than a test fluid contained in another metered fluid dose element 122. For example, a test fluid independently chosen for at least one metered fluid dose element 122 may be chosen to interact, react, activate, or de-activate another independently chosen test fluid in another metered fluid dose element 122.

Referring to FIGS. 1 and 2, the sample collection device 100 further includes an assay 150 disposed within the housing 102 and contacting the porous sample collection media 112. The assay 150 is configured to receive a fluid from the porous sample collection media 112. Specifically, the assay 150 is configured to receive the test fluid from the porous sample collection media 112. Therefore, the assay 150 is disposed in fluid communication with the porous sample collection media 112. Assay 150 qualitatively assesses or quantitatively measures the presence, amount, and/or functional activity of a the analyte on the sample collection media 112. The analyte can be a drug, biochemical substance, chemical element or compound, or cell in an organism or organic sample. Exemplary biological assays include, for example, PCR-ELISA or Fluorescence. Assay 150 can detect a molecule, often in low concentrations, that is a marker of disease or risk in the aerosol sample taken from the user/patient.

In some embodiments, the assay 150 is fixedly attached to the porous sample collection media 112. In some embodiments, the assay 150 is attached to the porous sample collection media 112 by an adhesive. In some embodiments, the adhesive is a porous adhesive and/or a medical grade adhesive. In some embodiments, the adhesive is continuous or discontinuous. In some embodiments, the adhesive is patterned. In some embodiments, the adhesive is a pressure sensitive adhesive (PSA). In some embodiments, the adhesive is structured and/or heat-activated. In some embodiments, the assay 150 is attached to the porous sample collection media 112 by hook and loop and/or 3M™ Dual Lock™ Reclosable Fasteners. In some embodiments, the assay 150 is attached to the porous sample collection media 112 by a means of mechanical attachment, such as pins, stapling, tongue and groove connections, etc.

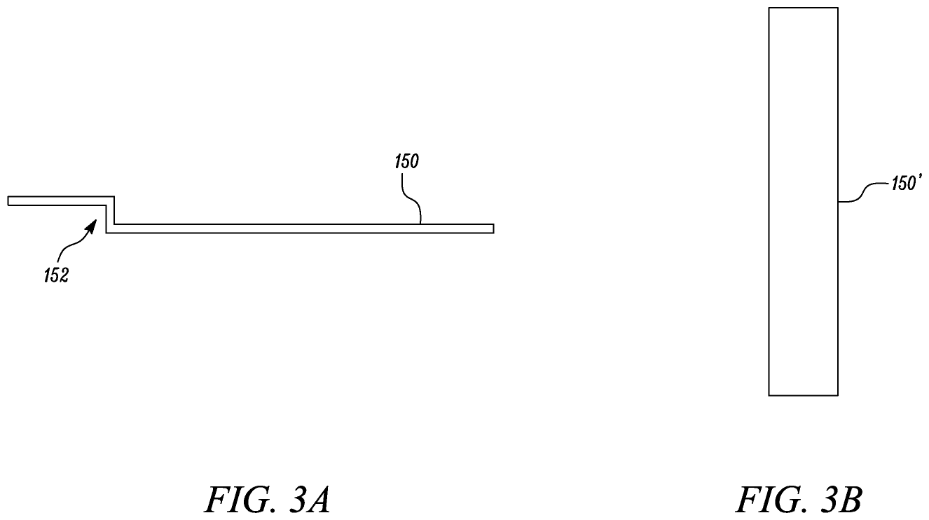

FIG. 3A illustrates a side view of the assay 150. Referring to FIGS. 1, 2 and 3A, in some embodiments, the assay 150 includes an L-shaped recess 152 configured to at least partially receive the porous sample collection media 112 therein. In some other embodiments, the assay 150 may include a recess of any other shape, such that the recess is configured to at least partially receive the porous sample collection media 112 therein.

Generally, the eluent is collected and tested by the assay 150. In some embodiments, the assay 150 detects virus or pathogen presence in the exhalation airflow 110 and/or the test fluid. In other words, the assay 150 first collects the eluent and then detects virus or pathogen presence in the eluent. Referring again to FIG. 1, the housing 102 further includes a display window 154 configured to allow visual inspection of at least a portion of the assay 150. Specifically, a user can see the test results on the assay 150, via the display window 154, and can get an indication of presence or absence of pathogens in the eluent and/or the test fluid.

In some embodiments, the assay 150 is a lateral flow assay. In the illustrated embodiment of FIGS. 1 and 2, the assay 150 may be oriented substantially parallel to the longitudinal axis “LA” of the housing 102. However, in some other embodiments, the assay 150 may be oriented at an oblique angle relative to the longitudinal axis “LA” of the housing 102.

FIG. 3B illustrates a front view of an assay 150′ according to another embodiment of the present disclosure. In some embodiments, the assay 150′ is a vertical flow assay. The assay 150′ may be used with the sample collection device 100 of FIGS. 1 and 2. The assay 150′ may contact the porous sample collection media 112 and configured to receive a fluid from the porous sample collection media 112. However, in contrast to the assay 150, the assay 150′ may be oriented substantially perpendicular to the longitudinal axis “LA” of the housing 102.

In general, lateral flow assays or vertical flow assays are paper-based platforms for the detection and quantification of analytes in complex mixtures, where a sample is placed on a test device and the results are displayed within 5-30 mins. Low development costs and ease of production of lateral flow assays have resulted in the expansion of its applications to multiple fields in which rapid tests are required. Lateral flow assay-based tests are widely used in hospitals, physician's offices and clinical laboratories for the qualitative and quantitative detection of specific antigens and antibodies, as well as products of gene amplification. A variety of biological samples can be tested using assays.

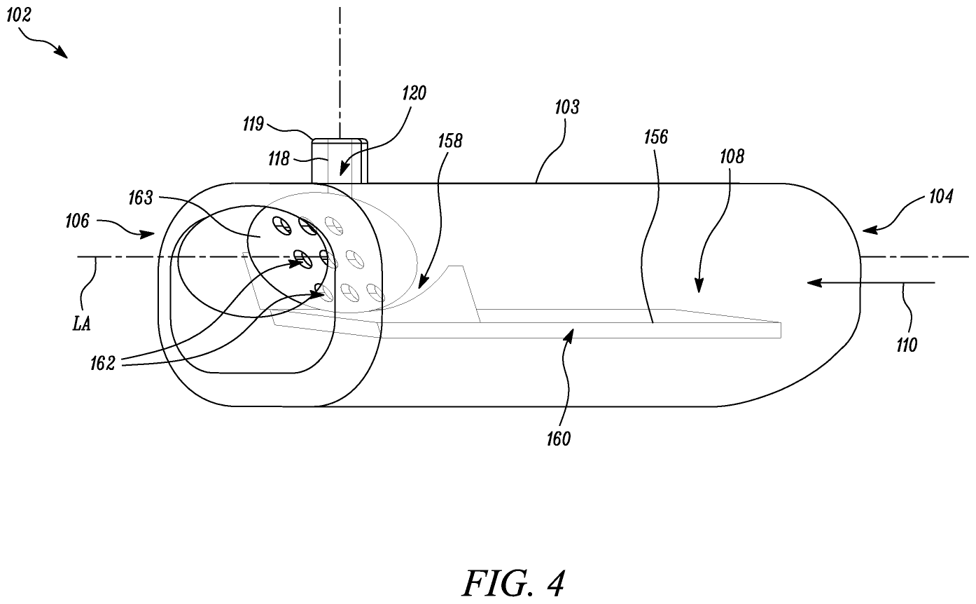

FIG. 4 illustrates the housing 102 according to an embodiment of the present disclosure. For illustration purpose, the housing 102 is shown transparent and some of the parts, such as the porous sample collection media 112 and the assay 150 are not shown in FIG. 3.

Referring to FIGS. 1, 2 and 4, in some embodiments, the housing 102 further includes a barrier 156 disposed between the fluid channel 108 and the assay 150. The barrier 156 is configured to prevent direct fluid communication between the exhalation airflow 110 and the assay 150. The barrier 156 may cover or enclose an entire area of the assay 150 to act as a layer between the fluid channel 108 and the assay 150.

Referring to FIG. 4, in some embodiments, the barrier 156 further includes an opening 158 configured to receive the porous sample collection media 112 therethrough. In some embodiments, the housing 102 further includes a receptacle 160 (also shown in FIGS. 1 and 2) disposed in fluid communication with the opening 158 and configured to receive the assay 150 therein. In other words, as the receptacle 160 is in fluid communication with the opening 158, the assay 150 is in fluid communication with the porous sample collection media 112 and receives the test fluid (and/or eluent) from the porous sample collection media 112.

With reference to FIGS. 1, 2 and 4, in some embodiments, the housing 102 further includes one or more vents 162 disposed in fluid communication with the fluid channel 108. The sample collection device 100 further includes a support element 163 at least partially disposed in the fluid channel 108 and configured to support the porous sample collection media 112 within the fluid channel 108. The support element 163 includes the one or more vents 162 therethrough. The support element 163 is disposed at an angle relative to the longitudinal axis “LA”. In some embodiments, the support element 163 is disposed, such that a major plane of the support element 163 is substantially parallel to the major plane 114 of the porous sample collection media 112. Further, the one or more vents 162 are disposed downstream of the porous sample collection media 112. In some embodiments, the one or more vents 162 are configured to allow egress of fluid from the fluid channel 108. Specifically, the one or more vents 162 are configured to allow egress of the exhalation airflow 110 from the fluid channel 108, via the second portion 106 of the housing 102.

Referring to FIGS. 1 and 2, the assay 150 is disposed within the housing 102 and fixedly attached to the porous sample collection media 112. In other words, a testing apparatus (i.e., the assay 150) is an integral part of the sample collection device 100. Hence, for testing a sample airflow for detection of pathogens, a testing apparatus is integrated in the sample collection device 100. Therefore, a single device (i.e., the sample collection device 100) can collect a sample airflow and test a corresponding eluent to detect presence of pathogens in the sample airflow. Moreover, the assay 150 is in contact with the porous sample collection media 112 to receive an eluent from the porous sample collection media 112. The sample collection device 100 including the assay 150 may therefore enable a rapid testing for detection of pathogens in a sample airflow. The sample collection device 100 including the assay 150 may further increase an efficiency and decrease an overall cost and a complexity of sample collection and subsequent sample testing. Moreover, the sample collection device 100 may minimize a possibility of contamination of the sample since both sample collection and testing are performed in a single unit. Further, the sample collection device 100 may be easily used by a user (e.g., a potential patient) without any prior training or professional help.

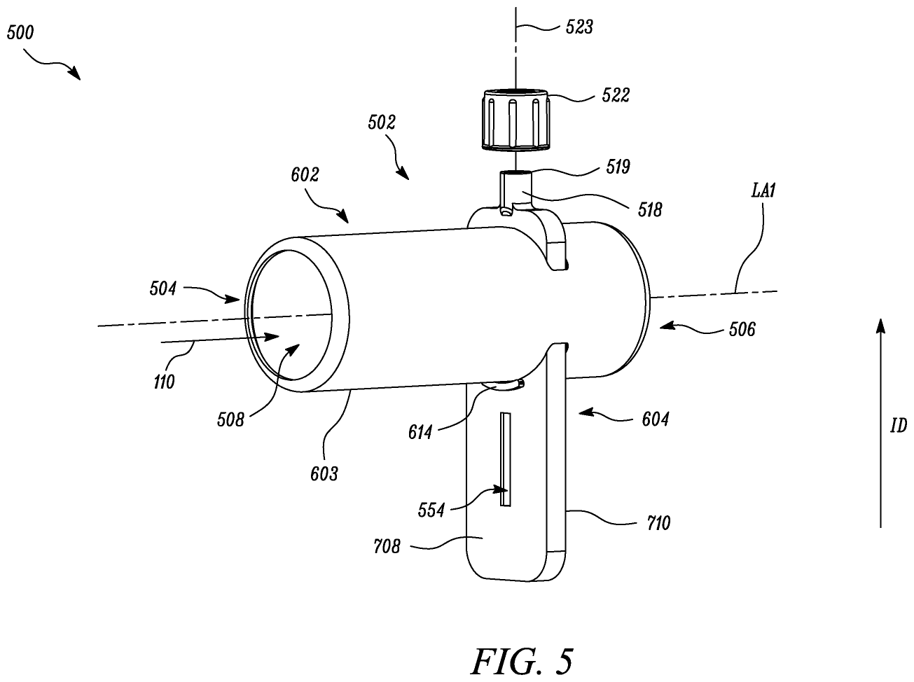

FIG. 5 illustrates a sample collection device 500 according to an embodiment of the present disclosure. The sample collection device 500 includes a housing 502 that includes a first housing part 602 and a second housing part 604 capable of engagement with the first housing part 602. The first housing part includes the air inlet and outlet. In some embodiments, the first housing is reusable. The second housing includes the sample collection media, the assay, and, where present, the fluid dose element. In some embodiments, the second housing is disposable.

As shown in FIG. 5, the first housing part 602 includes a first portion 504 and a second portion 506. Specifically, the first housing part 602 extends from the first portion 504 to the second portion 506. The first housing part 602 further includes a fluid channel 508 from the first portion 504 to the second portion 506. In some embodiments, one of the first portion 504 and the second portion 506 is a mouthpiece portion and the other of the first portion 504 and the second portion 506 is an air outlet portion. In the illustrated embodiment of FIG. 5, the first portion 504 is a mouthpiece portion and the second portion 506 is an air outlet portion.

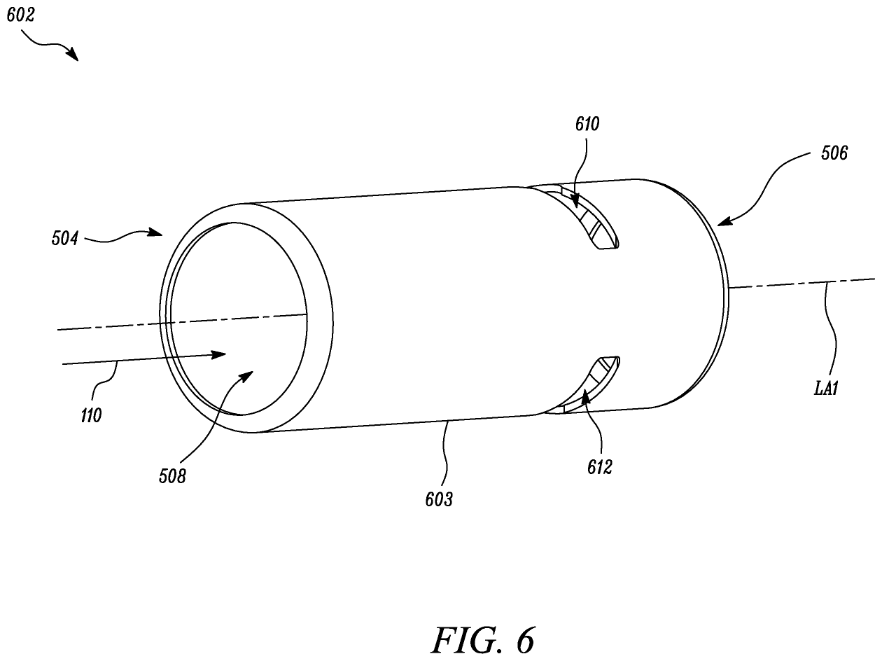

FIG. 6 illustrates a perspective view of the first housing part 602. The first portion 504 is configured to receive an exhalation airflow 110. In some other embodiments, the second portion 506 may receive the exhalation airflow 110. In that case, the first portion 504 is an air outlet portion and the second portion 506 is a mouthpiece portion. In the illustrated embodiment of FIG. 5, the fluid channel 508 is an air flow channel extending from the first portion 504 to the second portion 506. The fluid channel 508 extends longitudinally along a longitudinal axis “LA1” of the first housing part 602. The first housing part 602 may be formed of a rigid material, such as plastic.

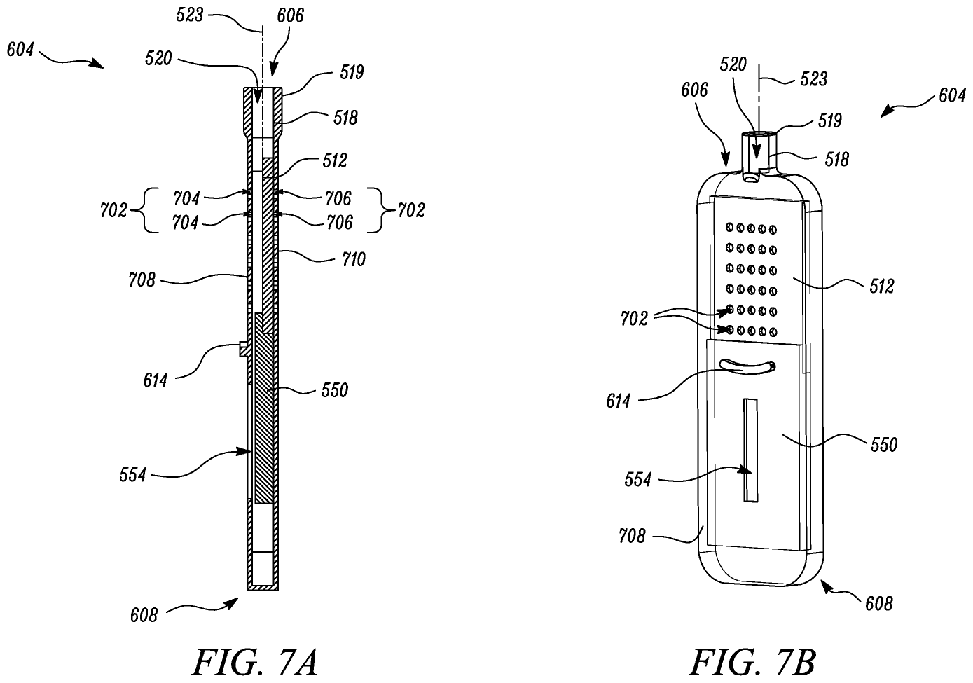

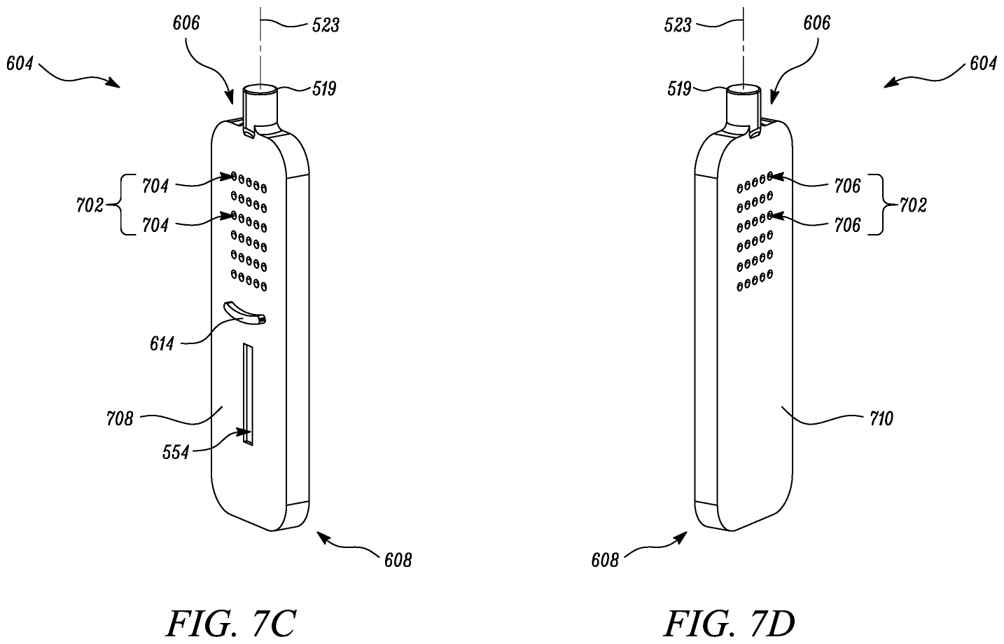

FIGS. 7A-7D illustrate different views of the second housing part 604 according to an embodiment of the present disclosure. In FIG. 7B, the second housing part 604 is shown transparent for illustrative purposes only. The sample collection device 500 further includes a porous sample collection media 512 disposed within the housing 502 and disposed in fluid communication with the fluid channel 508. Specifically, the porous sample collection media 512 is disposed within the second housing part 604. The porous sample collection media 512 is substantially similar to the porous sample collection media 112 illustrated in FIG. 1. The porous sample collection media 512 may be replaceable and changed out by a user, as desired. In an example, a user may exhale, via the first portion 504, into the sample collection device 500 and load the porous sample collection media 512 with a sample of the exhalation airflow 110 to form a loaded porous sample collection media 512. The user may then test the loaded porous sample collection media 512. The testing may take place with the loaded porous sample collection media 512 disposed in place within the sample collection device 500. After conducting the test, the user may replace the loaded porous sample collection media 512 with an unloaded porous sample collection media 512 within the sample collection device 500.

With continued reference to FIGS. 5 and 7A-7D, the sample collection device 500 further includes a fluid inlet port 518 defining a hole 520 through the housing 502 and disposed in fluid communication with the porous sample collection media 512. Specifically, the fluid inlet port 518 includes the hole 520 through the second housing part 604. In some embodiments, the second housing part 604 has a first end 606 and a second end 608 opposite to the first end 606. The second housing part 604 includes the fluid inlet port 518. The fluid inlet port 518 is disposed at the first end 606. The fluid inlet port 518 is adjacent to the porous sample collection media 512. In FIG. 7A, the sample collection media 512 is shown as generally perpendicular to airflow 110. However, sample collection media 512 can be aligned at an angle to airflow 110. In some embodiments, the fluid inlet port 518 includes a protrusion 519 extending away in a direction opposite to the porous sample collection media 512. The fluid inlet port 518 is configured to direct a test fluid onto the porous sample collection media 512.

In some embodiments, the sample collection device 500 further includes a metered fluid dose element 522 attached to the fluid inlet port 518. The metered fluid dose element 522 is substantially similar to the metered fluid dose element 122 illustrated in FIG. 1. The metered fluid dose element 522 is configured to dispense a metered dose of the test fluid into the fluid inlet port 518. In some embodiments, the metered fluid dose element 522 is movably attached to the fluid inlet port 518 via a threaded connection. In other words, the metered fluid dose element 522 is detachably connected to the fluid inlet port 518 of the second housing part 604. Specifically, the metered fluid dose element 522 is movably attached to the protrusion 519 defined by the fluid inlet port 518. Thus, the metered fluid dose element 522 may be moved between a fluid loaded position and a fluid depleted position by rotating the metered fluid dose element 522 about a threaded axis 523.

In other embodiments, the sample collection device 500 does not include a metered fluid dose element. Instead, fluid is dispensed by the user into/onto the sample collection device in the area shown as including the metered dose element 122. Fluid can be added or dispensed as is commonly known including by use of a dropper or bottle.

Upon engagement of the first housing part 602 and the second housing part 604, the metered fluid dose element 522 dispenses a metered dose of the test fluid through the loaded porous sample collection media 512 disposed in the fluid channel 508 to form an eluent. In other words, the test fluid may be delivered through the fluid inlet port 518 and applied onto the loaded porous sample collection media 512 to form the eluent. The eluent is then collected and tested, as described in the next paragraphs in the present disclosure.

With reference to FIGS. 5 and 7A-7D, the sample collection device 500 further includes an assay 550 disposed within the housing 502 and contacting the porous sample collection media 512. Specifically, the assay 550 is disposed within the second housing part 604. The porous sample collection media 512 and the assay 550 are disposed within the second housing part 604. The assay 550 is disposed proximal to the second end 608 of the second housing part 604. Thus, the porous sample collection media 512 is at least partly disposed between the fluid inlet port 518 and the assay 550.

The assay 550 is substantially similar to the assay 150 illustrated in FIG. 1. The assay 550 is configured to receive a fluid from the porous sample collection media 512. Specifically, the assay 550 is configured to receive the test fluid from the porous sample collection media 512. In some embodiments, the assay 550 is fixedly attached to the porous sample collection media 512. In some embodiments, the assay 550 is attached to the porous sample collection media 512 by an adhesive. In some embodiments, the assay 550 is attached to the porous sample collection media 512 by a means of mechanical attachment, such as pins, stapling, tongue and groove connections, etc.

Generally, the eluent is collected and tested by the assay 550. In some embodiments, the assay 550 detects virus or pathogen presence in the exhalation airflow 110 and/or the test fluid. In other words, the assay 550 first collects the eluent and then detects virus or pathogen presence in the eluent. Referring again to FIGS. 5 and 7A-7D, the housing 502 further includes a display window 554 configured to allow visual inspection of at least a portion of the assay 550. In other words, the second housing part 604 includes the display window 554 to allow visual inspection of at least a portion of the assay 550. Specifically, a user can see the test results on the assay 550, via the display window 554, and can get an indication of presence or absence of pathogens in the eluent and/or the test fluid.

Referring to FIG. 6, the first housing part 602 further includes a first slot 610 therethrough and a second slot 612 therethrough aligned with the first slot 610. With reference to FIGS. 5 and 6, in some embodiments, each of the first slot 610 and the second slot 612 is configured to slidably receive the second housing part 604 therethrough. In some embodiments, the second housing part 604 is slidably and removably received within the first housing part 602 along an insertion direction “ID”. In other words, upon engagement of the first housing part 602 and the second housing part 604, the second housing part 604 is at least partially received within the first housing part 602.

With reference to FIGS. 7A-7D, the second housing part 604 further includes a stop element 614 configured to abut the first housing part 602 and prevent further movement of the second housing part 604 relative to the first housing part 602 along the insertion direction “ID”. Specifically, when the second housing part 604 is slidably received within the first housing part 602, the stop element 614 abuts with an outer surface 603 of the first housing part 602 to prevent further movement of the second housing part 604 relative to the first housing part 602 along the insertion direction “ID”. In some embodiments, the stop element 614 may have a shape that generally corresponds to a shape of the outer surface 603 of the first housing part 602. For example, in case the outer surface 603 of the first housing part 602 has a tubular shape, the stop element 614 may have a curved shape with a radius of curvature substantially similar to a radius of curvature of the outer surface 603 of the first housing part 602.

In some embodiments, the first housing part 602 is detachably coupled to the second housing part 604. The first housing part 602 is detachably coupled to the second housing part 604 when the second housing part 604 is slidably received within the first housing part 602, and the metered fluid dose element 522 is movably attached with the fluid inlet port 518.

Referring to FIGS. 7A-7D, in some embodiments, the second housing part 604 further includes one or more apertures 702 disposed in fluid communication with the porous sample collection media 512. The one or more apertures 702 are configured to allow the exhalation airflow 110 to pass through the porous sample collection media 512. In other words, upon engagement of the first housing part 602 and the second housing part 604, the one or more apertures 702 allow the exhalation airflow 110 to impinge on the porous sample collection media 512 and to exit the second housing part 604 and the first housing part 602.

With reference to FIGS. 5-6 and 7A-7D, in some embodiments, the one or more apertures 702 include one or more first apertures 704 and one or more second apertures 706. In some embodiments, the second housing part 604 further includes a first wall 708 facing the first portion 504 and defining the one or more first apertures 704 therethrough. The second housing part 604 further includes a second wall 710 opposite to the first wall 708 and defining the one or more second apertures 706 therethrough. With reference to FIG. 7A, in some embodiments, each of the porous sample collection media 512 and the assay 550 is disposed between the first wall 708 and the second wall 710. Moreover, the stop element 614 is defined by the first wall 708 of the second housing part 604.

Therefore, upon engagement of the first housing part 602 and the second housing part 604, the one or more first apertures 704 allow the exhalation airflow 110 to pass therethrough and impinge on the porous sample collection media 512. Further, upon engagement of the first housing part 602 and the second housing part 604, the one or more second apertures 706 allow the exhalation airflow 110 to pass therethrough and exit the second housing part 604 and the first housing part 602.

In some embodiments, the porous sample collection media 512 is at least partly disposed between the one or more first apertures 708 and the assay 550 to prevent direct fluid communication between the exhalation airflow 110 and the assay 550. Specifically, when the first portion 504 is a mouthpiece portion, the porous sample collection media 512 is at least partly disposed between the one or more first apertures 708 and the assay 550 to prevent direct fluid communication between the exhalation airflow 110 and the assay 550. In some cases where the second portion 506 is a mouthpiece portion, the porous sample collection media 512 is at least partly disposed between the one or more second apertures 710 and the assay 550 to prevent direct fluid communication between the exhalation airflow 110 and the assay 550.

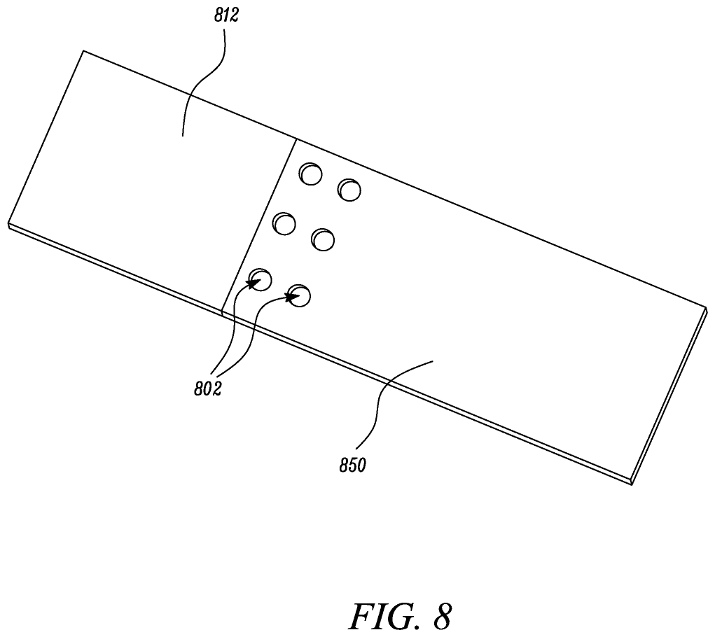

FIG. 8 shows a porous sample collection media 812 and an assay 850 that can be used in the sample collection device 500 illustrated in FIG. 5, according to an embodiment of the present disclosure. In some cases, the porous sample collection media 812 may be substantially similar to the porous sample collection media 512 illustrated in FIGS. 7A and 7B. In some other cases, the porous sample collection media 812 may have a different configuration from the porous sample collection media 512 illustrated in FIGS. 7A and 7B. However, the assay 850 further includes one or more apertures 802. By providing the one or more apertures 802 on the assay 850, an eluent from the porous sample collection media 812 flows through both of the major surface walls (only one major surface wall can be seen in FIG. 8) of the assay 850. While using the porous sample collection media 812 and the assay 850 in the sample collection device 500 (shown in FIG. 5), both of the major surface walls of the assay 850 may provide test results or indication of the presence or absence of pathogens in an eluent. Hence, while using the porous sample collection media 812 and the assay 850 in the sample collection device 500, the display window 554 provides a visual inspection of at least one of the major surface walls of the assay 850.

Therefore, when the first portion 504 is a mouthpiece portion during use of the sample collection device 500 including the assay 850, an eluent can be tested by visually inspecting one of the major surface walls of the assay 850. Further, when the second portion 506 is a mouthpiece portion during use of the sample collection device 500 including the assay 850, an eluent can be tested by visually inspecting the other of the major surface walls of the assay 850. Thus, a user can choose the first portion 504 as one of a mouthpiece portion and an air outlet portion while using the sample collection device 500 having the assay 850 and the porous sample collection media 812.

Referring to FIGS. 5-7D, the sample collection device 500 is a two-part assembly, i.e., including the first housing part 602 and the second housing part 604. The first housing part 602 and the second housing part 604 are removably coupled to each other. The sample collection device 500 may therefore facilitate a replacement of whole of the second housing part 604, whenever the porous sample collection media 512 and/or the assay 550 may need to be replaced. Thus, whenever there is a need for replacement, a user can easily replace a used porous sample collection media and/or a used assay from the sample collection device 500 with a new porous sample collection media and/or a new assay.

Further, in the sample collection device 500, the assay 550 is disposed within the second housing part 604 and fixedly attached to the porous sample collection media 512. Therefore, a single device (i.e., the sample collection device 500) can collect a sample airflow and test a corresponding eluent to detect presence of pathogens in the sample airflow. Moreover, the assay 550 is in contact with the porous sample collection media 512 to receive an eluent from the porous sample collection media 512. The sample collection device 500 including the assay 550 may therefore enable a rapid testing for detection of pathogens in a sample airflow. The sample collection device 500 including the assay 550 may further increase an efficiency and decrease an overall cost and a complexity of sample collection and subsequent sample testing. Moreover, the sample collection device 500 may minimize a possibility of contamination of the sample since both sample collection and testing are performed in a single unit. Since the second housing part 604 including the porous sample collection media 512 and the assay 550 is detachable from the first housing part 602, the first housing part 602 may be reused without the risk of cross-contamination between different samples. Further, the sample collection device 500 may be easily used by a user (e.g., a potential patient) without any prior training or professional help.

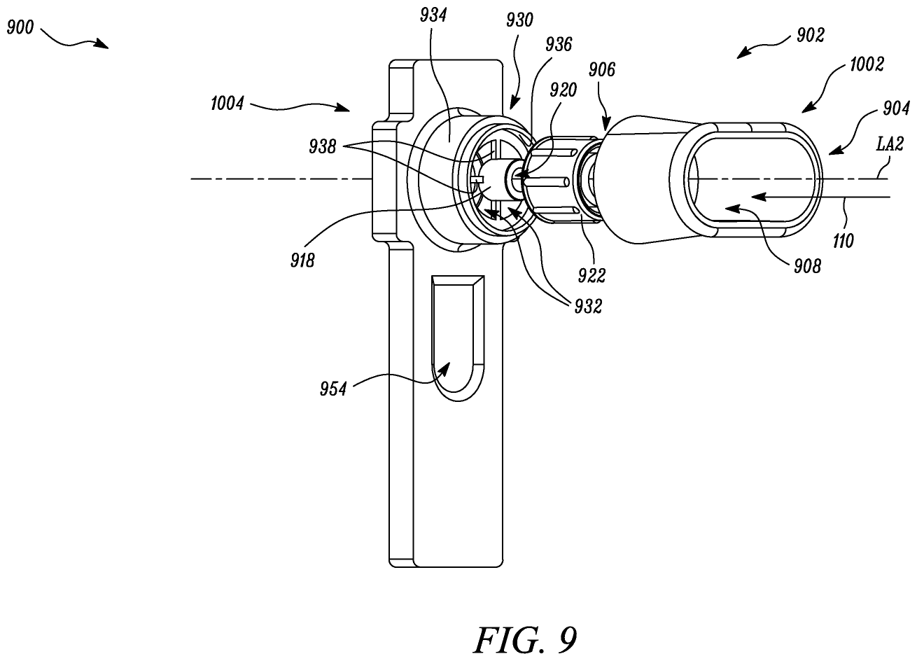

FIG. 9 illustrates a sample collection device 900 according to an embodiment of the present disclosure. The sample collection device 900 includes a housing 902. The housing 902 includes a first housing part 1002 and a second housing part 1004 engaged with the first housing part 1002. The first housing part 1002 includes a first portion 904 and a second portion 906. Specifically, the first housing part 1002 extends between the first portion 904 and the second portion 906. The first housing part 1002 further includes a fluid channel 908 from the first portion 904 to the second portion 906. In the illustrated embodiment of FIG. 9, the first portion 904 is a mouthpiece portion and the second portion 906 is an air outlet portion.

The first portion 904 is configured to receive an exhalation airflow 110. In the illustrated embodiment of FIG. 9, the fluid channel 908 is an air flow channel extending from the first portion 904 to the second portion 906. The fluid channel 908 extends longitudinally along a longitudinal axis “LA2” of the first housing part 1002. The first housing part 1002 may be formed of a rigid material, such as plastic.

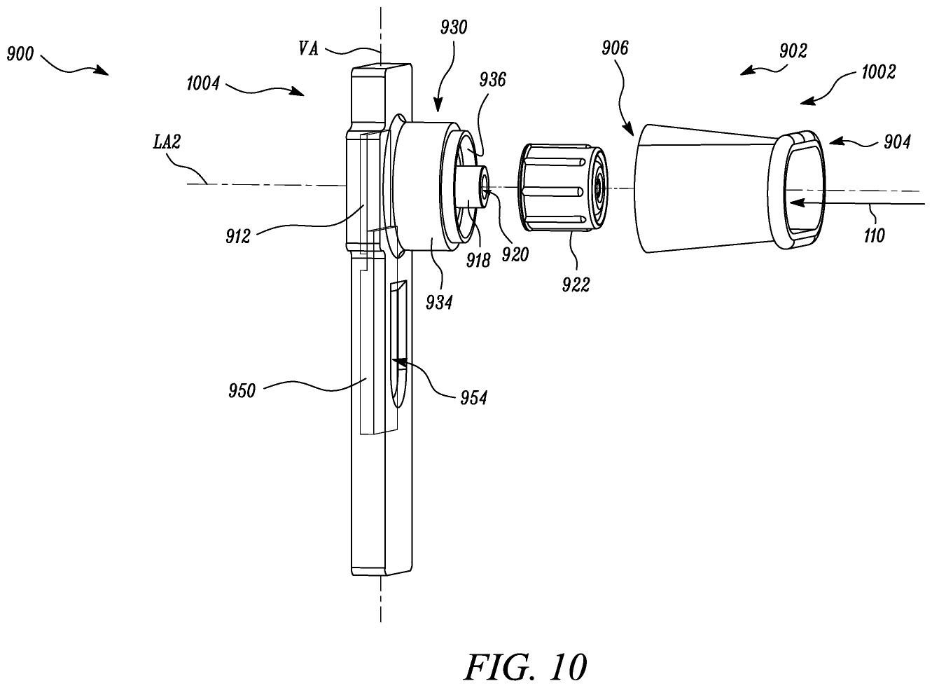

FIG. 10 illustrates another view of the sample collection device 900, where the second housing part 1004 is shown transparent for illustrative purposes only. The sample collection device 900 further includes a porous sample collection media 912 disposed within the housing 902 and disposed in fluid communication with the fluid channel 908. Specifically, the porous sample collection media 912 is disposed within the second housing part 1004. The porous sample collection media 912 is substantially similar to the porous sample collection media 512 illustrated in FIGS. 7A and 7B. The porous sample collection media 912 may be replaceable and changed out by a user, as desired. In an example, a user may exhale, via the first portion 904, into the sample collection device 900 and load the porous sample collection media 912 with a sample of the exhalation airflow 110 to form a loaded porous sample collection media 912. The user may then test the loaded porous sample collection media 912. The testing may take place with the loaded porous sample collection media 912 disposed in place within the sample collection device 900. After conducting the test, the user may replace the loaded porous sample collection media 912 with an unloaded porous sample collection media 912 within the sample collection device 900.

With reference to FIGS. 9 and 10, the sample collection device 900 further includes a fluid inlet port 918 defining a hole 920 through the housing 902 and disposed in fluid communication with the porous sample collection media 912. Specifically, the fluid inlet port 918 includes the hole 920 through the second housing part 1004. The fluid inlet port 918 is adjacent to the porous sample collection media 912. The fluid inlet port 918 is configured to direct a test fluid onto the porous sample collection media 912.

In some embodiments, the sample collection device 900 further includes a metered fluid dose element 922 attached to the fluid inlet port 918. The metered fluid dose element 922 is substantially similar to the metered fluid dose element 522 illustrated in FIG. 5. The metered fluid dose element 922 is configured to dispense a metered dose of the test fluid into the fluid inlet port 918. In some embodiments, the metered fluid dose element 922 is movably attached to the fluid inlet port 918 via a threaded connection. In other words, the metered fluid dose element 922 is detachably connected to the fluid inlet port 918 of the second housing part 1004. Thus, the metered fluid dose element 922 may be moved between a fluid loaded position and a fluid depleted position by rotating the metered fluid dose element 922 about a threaded axis (i.e., the longitudinal axis “LA2” of the first housing part 1002).

In other embodiments, the sample collection device 900 does not include a metered fluid dose element. Instead, fluid is dispensed by the user into/onto the sample collection device in the area shown as including the metered dose element 122. Fluid can be added or dispensed as is commonly known including by use of a dropper or bottle.

In some embodiments, the second housing part 1004 further includes a coupling portion 930 including the fluid inlet port 918. The coupling portion 930 is configured to be interchangeably and removably coupled with the first housing part 1002 and the metered fluid dose element 922. Therefore, at one time, the coupling portion 930 is coupled to only one of the first housing part 1002 and the metered fluid dose element 922. Upon coupling of the first housing part 1002 with the coupling portion 930, a user may exhale the exhalation airflow 110 into the sample collection device 900 via the first portion 904. In other words, upon coupling of the first housing part 1002 with the coupling portion 930, a user may exhale, via the first portion 904, into the sample collection device 900 and load the porous sample collection media 912 with a sample of the exhalation airflow 110 to form a loaded porous sample collection media 912.

Referring to FIG. 9, in some embodiments, the coupling portion 930 further includes one or more apertures 932 of the second housing part 1004. The one or more apertures 932 are disposed in fluid communication and aligned with the fluid inlet port 918. In some embodiments, the fluid channel 908 of the first housing part 1002 is disposed in fluid communication with the one or more apertures 932 upon coupling of the first housing part 1002 with the coupling portion 930.

In some embodiments, the one or more apertures 932 are configured to direct the test fluid from the fluid inlet port 918 to the porous sample collection media 912 when the metered fluid dose element 922 (where present) is coupled with the coupling portion 930. In other words, upon coupling of the metered fluid dose element 922 (where present) with the coupling portion 930, the metered fluid dose element 922 (where present) dispenses a metered dose of the test fluid into the fluid inlet port 918 via the one or more apertures 932. The metered dose of the test fluid passes through the loaded porous sample collection media 912 to form an eluent. In other words, the test fluid may be delivered through the fluid inlet port 918 and applied onto the loaded porous sample collection media 912 to form the eluent. The eluent is then collected and tested, as described in the next paragraphs in the present disclosure.

In the illustrated embodiment, the one or more apertures 932 includes multiple apertures 932 angularly spaced apart from each other with respect to the longitudinal axis “LA2”. However, the coupling portion 930 may include any number of apertures 932 disposed in any suitable configuration as per application requirements.

With reference to FIGS. 9 and 10, the coupling portion 930 further includes a shoulder 934 configured to be detachably coupled with the first housing part 1002. In other words, the shoulder 934 is detachably coupled to the first housing part 1002 upon coupling of the first housing part 1002 with the coupling portion 930 of the second housing part 1004.

In some embodiments, the coupling portion 930 further includes an internal surface 936 and one or more support members 938. The internal surface 936 at least partially includes the one or more apertures 932. Further, the one or more support members 938 connect the fluid inlet port 918 with the internal surface 936. The one or more support members 938 may be elongate members that connect the fluid inlet port 918 with the internal surface 936. In the illustrated embodiment, the coupling portion 930 includes multiple support members 938 angularly spaced apart from each other relative to the longitudinal axis “LA2”. Each aperture 932 may be at least partially defined between corresponding adjacent support members 938.

The sample collection device 900 further includes an assay 950 disposed within the housing 902 and contacting the porous sample collection media 912. Specifically, the assay 950 is disposed within the second housing part 1004. The porous sample collection media 912 and the assay 950 are disposed within the second housing part 1004. The porous sample collection media 912 is at least partly disposed between the fluid inlet port 918 and the assay 950.

The assay 950 is substantially similar to the assay 950 illustrated in FIGS. 7A and 7B. The assay 950 is configured to receive a fluid from the porous sample collection media 912. Specifically, the assay 950 is configured to receive the test fluid from the porous sample collection media 912. In some embodiments, the assay 950 is fixedly attached to the porous sample collection media 912. In some embodiments, the assay 950 is attached to the porous sample collection media 912 by an adhesive. In some embodiments, the assay 950 is attached to the porous sample collection media 912 by a means of mechanical attachment, such as pins, stapling, tongue and groove connections, etc.

Generally, the eluent is collected and tested by the assay 950. In some embodiments, the assay 950 detects virus or pathogen presence in the exhalation airflow 110 and/or the test fluid. In other words, the assay 950 first collects the eluent and then detects virus or pathogen presence in the eluent. Referring to FIG. 9, the housing 902 further includes a display window 954 configured to allow visual inspection of at least a portion of the assay 950. In other words, the second housing part 1004 includes the display window 954 to allow visual inspection of at least a portion of the assay 950. Specifically, a user can see the test results on the assay 950, via the display window 954, and can get an indication of the presence or absence of pathogens in the eluent and/or the test fluid.

In some embodiments, the second housing part 1004 includes a longitudinal axis “VA” (shown in FIG. 10) along its length. The longitudinal axis “VA” of the second housing part 1004 may be orthogonal to the longitudinal axis “LA2” of the first housing part 1002. In some embodiments, the assay 950 is spaced apart from the one or more apertures 932 along the longitudinal axis “VA” to prevent direct fluid communication between the exhalation airflow 110 and the assay 950. Specifically, a gap may be provided between the one or more apertures 932 and the assay 950 along the longitudinal axis “VA” of the second housing part 1004 to prevent direct fluid communication between the exhalation airflow 110 and the assay 950.

Referring to FIGS. 9 and 10, the sample collection device 900 is a two-part assembly, i.e., including the first housing part 1002 and the second housing part 1004. The first housing part 1002 and the second housing part 1004 are removably coupled to each other. The sample collection device 900 may therefore facilitate a replacement of whole of the second housing part 1004, whenever the porous sample collection media 912 and/or the assay 950 may need to be replaced. Thus, whenever there is a need for replacement, a user can easily replace a used porous sample collection media and/or a used assay from the sample collection device 900 with a new porous sample collection media and/or a new assay.

Further, in the sample collection device 900, the assay 950 is disposed within the second housing part 1004 and fixedly attached to the porous sample collection media 912. Therefore, a single device (i.e., the sample collection device 500) can collect a sample airflow and test a corresponding eluent to detect presence of pathogens in the sample airflow. Moreover, the assay 950 is in contact with the porous sample collection media 912 to receive an eluent from the porous sample collection media 912. The sample collection device 900 including the assay 950 may enable a rapid testing for detection of pathogens in a sample airflow. The sample collection device 900 including the assay 950 may further increase an efficiency and decrease an overall cost and a complexity of sample collection and subsequent sample testing. Moreover, the sample collection device 900 may minimize a possibility of contamination of the sample since both sample collection and testing are performed in a single unit. Since the second housing part 1004 including the porous sample collection media 912 and the assay 950 is detachable from the first housing part 1002, the first housing part 1002 may be reused without the risk of cross-contamination between different samples. Further, the sample collection device 900 may be easily used by a user (e.g., a potential patient) without any prior training or professional help.

For using the sample collection device 900 to detect the presence of pathogens in a sample airflow, a user first couples the first housing part 1002 with the second housing part 1004. Further, the user exhales, via the first housing part 1002, a sample airflow into the sample collection device 900. The sample airflow is impinged onto the porous sample collection media 912 (in fluid communication with the fluid inlet port 918) to form a loaded porous sample collection media 912. The user then uncouples the first housing part 1002 from the second housing part 1004, and then couples the second housing part 1004 with the metered fluid dose element 922. A test fluid is dispensed by the metered fluid dose element 922 into the fluid inlet port 918. As the fluid inlet port 918 is disposed in fluid communication with the porous sample collection media 912, an eluent is formed at the loaded porous sample collection media 912 and the assay 950 receives the eluent from the loaded porous sample collection media 912. At least a part of the assay 950 is visually inspected via the display window 954 to detect the presence or absence of pathogens in the eluent.

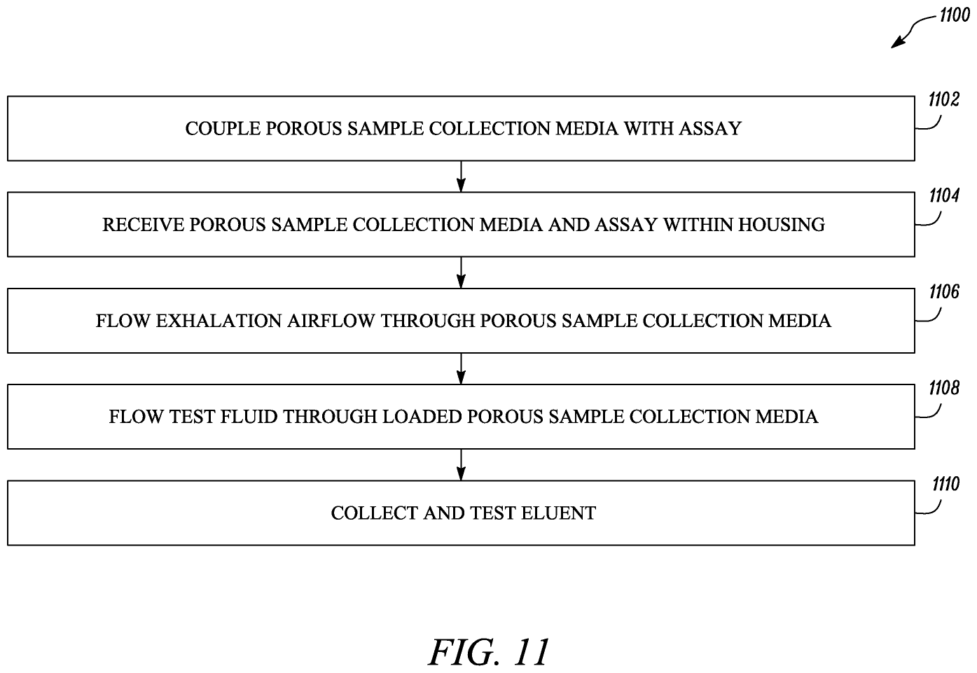

FIG. 11 illustrates a flow chart for a method 1100 for testing the exhalation airflow 110 in the sample collection devices 100, 500, 900. At step 1102, the method 1100 includes coupling the porous sample collection media 112, 512, 912 with the respective assays 150, 550, 950. Referring to FIGS. 1, 7A, and 10, the assays 150, 550, 950 are configured to receive a fluid from the respective porous sample collection media 112, 512, 912. Specifically, the assays 150, 550, 950 are configured to receive a test fluid from the respective porous sample collection media 112, 512, 912.

With reference to FIGS. 1, 7A, and 10, at step 1104, the method 1100 further includes receiving the porous sample collection media 112, 512, 912 and the respective assays 150, 550, 950 within the respective housings 102, 502, 902. Referring to FIGS. 1, 5, and 9, the housings 102, 502, 902 define the respective fluid channels 108, 508, 908 to receive the exhalation airflow 110.

Referring to FIGS. 1, 5, and 10, at step 1106, the method 1100 includes flowing the exhalation airflow 110 through the porous sample collection media 112, 512, 912. The porous sample collection media 112, 512, 912 are disposed in fluid communication with the respective fluid channels 108, 508, 908 and form the respective loaded porous sample collection media 112, 512, 912.

Referring to FIGS. 1, 5, and 9, at step 1108, the method 1100 includes flowing, a test fluid through the respective loaded porous sample collection media 112, 512, 912 disposed in the respective fluid channels 108, 508, 908 forming an eluent. In some embodiments, this is done by way of a metered fluid dose element. At step 1110, the method 1100 includes collecting and testing, by the assays 150, 550, 950, the eluent.

In some embodiments, flowing the test fluid includes flowing a metered dose in a range from 50 microliters to 400 microliters of the test fluid through the loaded porous sample collection media 112, 512, 912 disposed in the respective fluid channels 108, 508, 908.

In some embodiments, testing the eluent includes detecting a presence of virus or pathogen in the eluent.

Referring to FIGS. 2, 5, and 9, in some embodiments, the method 1100 further includes detachably connecting the metered fluid dose elements 122, 522, 922 to the respective fluid inlet ports 118, 518, 918 of the respective housings 102, 502, 902.

Referring to FIG. 5, in some embodiments, the housing 502 includes a first housing part 602 defining the fluid channel 508 and a second housing part 604 separate from the first housing part 602. As shown in FIGS. 7A and 7B, the second housing part 604 is configured to receive the porous sample collection media 512 and the assay 550 therein. Referring to FIGS. 9 and 10, the housing 902 includes a first housing part 1002 defining the fluid channel 908 and a second housing part 1004 separate from the first housing part 1002. The second housing part 1004 is configured to receive the porous sample collection media 912 and the assay 950 therein.

Referring to FIG. 5, in some embodiments, the method 1100 further includes detachably coupling the first housing part 602 with the second housing part 604. The first housing part 602 is detachably coupled with the second housing part 604 when the second housing part 604 is slidably received within the first housing part 602, and the metered fluid dose element 522 is movably attached with the fluid inlet port 518.

Referring to FIGS. 9 and 10, in some embodiments, the method 1100 further includes detachably coupling the first housing part 1002 with the second housing part 1004. Specifically, the first housing part 1002 is detachably coupled with the second housing part 1004 by coupling the shoulder 934 of the coupling portion 930 of the second housing part 1004 to the first housing part 1002.

Referring again to FIGS. 9 and 10, in some embodiments, the method 1100 further includes removing the first housing part 1002 from the second housing part 1004. In other words, the method 1100 includes uncoupling the first housing part 1002 from the second housing part 1004 after a user exhales a sample airflow into the sample collection device 900. When the user exhales the sample airflow into the sample collection device 900, the sample airflow is impinged onto the porous sample collection media 912 (in fluid communication with the fluid inlet port 918) to form a loaded porous sample collection media 912. The method 1100 further includes detachably coupling the metered fluid dose element 922 to the second housing part 1004. A test fluid is dispensed by the metered fluid dose element 922 into the fluid inlet port 918. As the fluid inlet port 918 is disposed in fluid communication with the porous sample collection media 912, an eluent is formed at the loaded porous sample collection media 912 and the assay 950 receives the eluent from the loaded porous sample collection media 912. At least a part of the assay 950 is visually inspected via the display window 954 to detect the presence or absence of pathogens in the eluent.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A sample collection device comprising:
a housing extending from a first portion to a second portion, the housing defining a fluid channel from the first portion to the second portion, wherein the first portion is configured to receive an exhalation airflow;
a porous sample collection media comprising a nonwoven filtration layer and disposed within the housing and in fluid communication with the fluid channel;
a fluid inlet port defining a hole through the housing and disposed in fluid communication with the porous sample collection media, wherein the fluid inlet port is configured to direct a test fluid onto the porous sample collection media;
a metered fluid dose element movably attached to the fluid inlet port a configured to dispense a metered dose of the test fluid into the fluid inlet port; and
an assay disposed within the housing and contacting the porous sample collection media, wherein the assay is configured to receive fluid from the porous sample collection media.

2. The sample collection device of claim 1, wherein the housing further comprises a screen disposed between the fluid channel and the assay, and wherein the screen is configured to prevent direct fluid communication between the exhalation airflow and the assay.

3. The sample collection device of claim 1, further comprising:
a metered fluid dose element attached to the fluid inlet port, wherein the metered fluid dose element is configured to dispense a metered dose of the test fluid into the fluid inlet port.

4. (canceled)

5. The sample collection device of claim 1, wherein the housing comprises:
a first housing part comprising the first portion and the second portion, the first housing part further defining the fluid channel from the first portion to the second portion; and
a second housing part engaged with the first housing part and comprising the fluid inlet port, wherein the porous sample collection media and the assay are disposed within the second housing part, the second housing part further defining one or more apertures disposed in fluid communication with the porous sample collection media and configured to allow the exhalation airflow to pass through the porous sample collection media.

6-14. (canceled)

15. The sample collection device of claim 1, wherein the housing further comprises a display window configured to allow visual inspection of at least a portion of the assay.

16. The sample collection device of claim 1, further comprising a screen disposed in the housing and upstream of the porous sample collection media, wherein the screen includes one or more flow apertures therethrough.

17. (canceled)

18. The sample collection device of claim 1, wherein the fluid inlet port comprises a protrusion extending away from an outer surface of the housing.

19. The sample collection device of claim 1, wherein the metered fluid dose element is movably attached to the fluid inlet port via a threaded connection.

20. (canceled)

21. The sample collection device of claim 1, wherein the porous sample collection media includes a surface area and the metered fluid dose element comprises a fluid reservoir having a volume, and wherein the volume divided by the surface area is in a range from 10 microliters/cm² to 400 microliters/cm², or from 10 microliters/cm² to 250 microliters/cm².

22. The sample collection device of claim 21, wherein the volume of the fluid reservoir is in a range from 50 microliters to 500 microliters.

23. The sample collection device of claim 1, wherein the porous sample collection media comprising the nonwoven filtration layer has an electrostatic charge.

24. (canceled)

25. The sample collection device claim 1, wherein the test fluid is at least one of an aqueous fluid, an aqueous buffer solution, an aqueous fluid including a surfactant, a saline solution, or a saline solution including a surfactant.

26. (canceled)

27. The sample collection device of claim 1, wherein the assay is fixedly attached to the porous sample collection media.

28. The sample collection device of claim 1, wherein the assay is configured to detect virus or pathogen presence in the exhalation airflow.

29. The sample collection device of claim 1, wherein the assay is a lateral flow assay.

30. The sample collection device of claim 1, wherein the assay is a vertical flow assay.

31. The sample collection device of claim 5, wherein one of the first portion and the second portion is a mouthpiece portion and the other of the first portion and the second portion is an air outlet portion.

32. A method for testing an exhalation airflow, the method comprising:
coupling a porous sample collection media with an assay, such that the assay is configured to receive a fluid from the porous sample collection media, the porous sample collection media comprising a nonwoven filtration layer;
receiving the porous sample collection media and the assay within a housing, the housing defining a fluid channel configured to receive the exhalation airflow;
flowing the exhalation airflow through the porous sample collection media, wherein the porous sample collection media is disposed in fluid communication with the fluid channel and forms a loaded porous sample collection media;
flowing a metered dose of 50 microliters to 400 microliters of a test fluid through the loaded porous sample collection media disposed in the fluid channel forming an eluent; and
collecting and testing, by the assay, the eluent.

33. (canceled)

34. The method of claim 32, wherein testing the eluent comprises detecting a presence of virus or pathogen in the eluent.

35. The method of claim 32, further comprising a metered fluid dose element that is detachably connected to a fluid inlet port of the housing.

36-38. (canceled)
